(19) United States
(12) Patent Application Publication
(10) Pub. No.: US 2013/0109948 A1
(43) Pub. Date: May 2, 2013

(54) METHODS AND SYSTEMS FOR DETERMINING PHYSIOLOGICAL PARAMETERS USING TWO PHOTOACOUSTIC PEAKS

(71) Applicant: Youzhi Li, Longmont, CO (US)

(72) Inventor: Youzhi Li, Longmont, CO (US)

(73) Assignee: Nellcor Puritan Bennett LLC, Boulder, CO (US)

(21) Appl. No.: 13/284,545

(22) Filed: Oct. 28, 2011

(51) Int. Cl. A61B 8/00; A61B 6/00; A61B 5/145

(52) U.S. Cl. 600/407

(57) ABSTRACT

A patient monitoring system may use photoacoustic sensing to determine one or more physiological parameters of a subject. The system may detect an acoustic pressure, response generated by the application and absorption of light, which may include two peaks. The peaks may correspond to surfaces of a feature such as a blood vessel, and the peak information may allow determination of physiological information. For example, the two peaks may be analyzed and an effective attenuation coefficient may be determined, from which hemoglobin concentration, blood oxygen saturation, or other physiological parameters may be derived.

The present disclosure relates to determining physiological parameters based on a photoacoustic response, and more particularly relates to determining physiological parameters based on peak information of two peaks of a photoacoustic response.

### SUMMARY

A physiological monitoring system may be configured to determine a physiological parameter using photoacoustic analysis. In some embodiments, the system may include a light source, configured to provide a photonic signal at a suitable wavelength to a feature of a subject, causing an acoustic pressure response of the subject via the photoacoustic effect. The feature may be, for example, a blood vessel of the subject. The system may include an acoustic detector configured to detect an acoustic pressure signal at one or more locations of the subject.

In some embodiments, the system may determine a first peak and a second peak in the acoustic pressure signal indicative of a boundary of the feature. The first peak may correspond to a front boundary of the feature, and the second peak may correspond to a back boundary of the feature, and the peaks may occur sequentially. In some embodiments, the system may determine a first time associated with the first peak and a second time associated with the second peak, and may further determine a difference between the second time and the first time. The system may determine a physiological parameter of the subject such as, for example, pulse rate, an arterial blood oxygen saturation value, a venous blood oxygen saturation value, and hemoglobin concentration, based at least in part on the first and second peaks.

In some embodiments, the system may divide the acoustic pressure signal at the first time by the acoustic pressure signal to the photonic signal at the second time to generate a ratio. The ratio may allow some variables to be cancelled, and may allow an effective attenuation coefficient to be determined. In some embodiments, one or more physiological parameters of the subject may be determined based at least in part on the effective attenuation coefficient.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system, in accordance with some embodiments of the present disclosure;

FIG. 2 is a block diagram of the illustrative patient monitoring system of FIG. 1 coupled to a patient, in accordance with some embodiments of the present disclosure;

FIG. 3 shows a block diagram of an illustrative signal processing system, in accordance with some embodiments of the present disclosure;

FIG. 4 is an illustrative photoacoustic arrangement, in accordance with some embodiments of the present disclosure;

FIG. 5 is a plot of an illustrative photoacoustic signal, including two peaks, in accordance with some embodiments of the present disclosure; and

FIG. 6 is a flow diagram of illustrative steps for determining a physiological parameter based at least in part on two peaks of an acoustic pressure signal, in accordance with some embodiments of the present disclosure.

### DETAILED DESCRIPTION OF THE FIGURES

Photoacoustics (or “optoacoustics”) or the photoacoustic effect (or “optoacoustic effect”) refers to the phenomenon in which one or more wavelengths of light are presented to and absorbed by one or more constituents of an object, thereby causing an increase in kinetic energy of the one or more constituents, which causes an associated pressure response within the object. Particular modulations or pulsing of the incident light, along with measurements of the corresponding pressure response in, for example, tissue of the subject, may be used for medical imaging, physiological parameter determination, or both. For example, the concentration of a constituent, such as hemoglobin (e.g., oxygenated, deoxygenated and/or total hemoglobin) may be determined using photoacoustic analysis.

A photoacoustic system may include a photoacoustic sensor that is placed at a site on a subject, typically the wrist, neck, forehead, temple, or anywhere an artery is accessible noninvasively. In some embodiments, the photoacoustic techniques described herein are used to monitor large blood vessels, such as a major artery or vein near the heart. The photoacoustic system may use a light source, and any suitable light guides (e.g., fiber optics), to pass light through the subject's tissue, or a combination of tissue thereof (e.g., organs), and an acoustic detector to sense the pressure response of the tissue induced by the light absorption by a blood vessel. Tissue may include muscle, fat, blood, blood vessels, and/or any other suitable tissue types. In some embodiments, the light source may be a laser or laser diode, operated in pulsed or continuous wave (CW) mode. In some embodiments, the acoustic detector may be an ultrasound detector, which may be suitable to detect pressure fluctuations arising from the constituent's absorption of the incident light of the light source.

In some embodiments, the light from the light source may be focused, shaped, or otherwise spatially modulated to illuminate a particular region of interest. In some arrangements, photoacoustic monitoring may allow relatively higher spatial resolution than line of sight optical techniques (e.g., path integrated absorption measurements). The enhanced spatial resolution of the photoacoustic technique may allow for imaging, scalar field mapping, and other spatially resolved results, in 1, 2, or 3 spatial dimensions. The acoustic response to the photonic excitation may radiate from the illuminated region of interest, and accordingly may be detected at multiple positions.

The photoacoustic system may measure the pressure response that is received at the acoustic sensor as a function of time. The photoacoustic system may also include sensors at multiple locations. A signal representing pressure versus time or a mathematical manipulation of this signal (e.g., a scaled version thereof, etc.) may be referred to as the photoacoustic (PA) signal. The PA signal may be used to calculate any of a number of physiological parameters, including an amount of a blood constituent (e.g., oxy-hemoglobin), at a particular spatial location. In some embodiments, PA signals from multiple spatial locations may be used to construct an image (e.g., imaging blood vessels) or a scalar field (e.g., a hemoglobin concentration field).

In some applications, the light passed through the tissue is selected to be of one or more wavelengths that are absorbed by the constituent in an amount representative of the amount of the constituent present in the tissue. The absorption of light passed through the tissue varies in accordance with the amount of the constituent in the tissue. For example, Red and/or infrared (IR) wavelengths may be used because highly oxygenated blood will absorb relatively less Red light and more IR light than blood with a lower oxygen saturation.

Any suitable light source may be used, and characteristics of the light provided by the light source may be controlled in any suitable manner. In some embodiments, a pulsed light source may be used to provide relatively short-duration pulses (e.g., nanosecond pulses) of light to the region of interest. Accordingly, the use of a pulse light source may result in a relatively broadband acoustic response (e.g., depending on the pulse duration). The use of a pulsed light source will be referred to herein as the “Time Domain Photoacoustic” (TD-PA) technique. A convenient starting point for analyzing a TD-PA signal is given by Eq. 1:

p(z)=Γμₐφ(z) (1)

under conditions where the irradiation time is small compared to the characteristic thermal diffusion time determined by the properties of the specific tissue type. Referring to Eq. 1, p(z) is the PA signal (indicative of the maximum induced pressure rise) at spatial location z indicative of acoustic pressure, Γ is the dimensionless Gruneisen parameter of the tissue, μₐ is the effective absorption coefficient of the tissue (or constituent thereof) to the incident light, and φ(z) is the optical fluence at spatial location z. The Grüneisen parameter is a dimensionless description of thermoelastic effects, and may be illustratively formulated by Eq. 2:

$$Γ = \frac{β {c}_{a}^{2}}{{C}_{P}} \qquad ( 2 )$$

where Cₐ² is the speed of sound in the tissue, β is the isobaric volume thermal expansion coefficient, and C_{P} is the specific heat at constant pressure. In some circumstances, the optical fluence, at spatial location z (within the subject's tissue) of interest may be dependent upon the light source, the location itself (e.g., the depth), and optical properties (e.g., scattering coefficient, absorption coefficient, or other properties) along the optical path. For example, Eq. 3 provides an illustrative expression for the attenuated optical fluence at a depth z:

φ(z)=φ₀e^{-μ}^{_{eff}}^{z} (3)

where φ₀ is the optical fluence from the light source incident at the tissue surface, z is the path length (i.e., the depth into the tissue in this example), and μ_{eff} is an effective attenuation coefficient of the tissue along the path length in the tissue in this example.

In some embodiments, a more detailed expression or model may be used rather than the illustrative expression of Eq. 3. In some embodiments, the actual pressure encountered by an acoustic detector may be proportional to Eq. 1, as the focal distance and solid angle (e.g., face area) of the detector may affect the actual measured PA signal. In some embodiments, an ultrasound detector positioned relatively farther away from the region of interest, will encounter a relatively smaller acoustic pressure. For example, the acoustic pressure received at a circular area A_{d} positioned at a distance R from the illuminated region of interest may be given by Eq. 4:

p_{d}=p(z)f(rₛ,R,A_{d}) (4)

where rₛ is the radius of the illuminated region of interest (and typically rₛ<R), and p(z) is given by Eq. 1. In some embodiments, the detected acoustic pressure amplitude may decrease as the distance R increases (e.g., for a spherical acoustic wave).

In some embodiments, a modulated CW light source may be used to provide a photonic excitation of a tissue constituent to cause a photoacoustic response in the tissue. The CW light source may be intensity modulated at one or more characteristic frequencies. The use of a CW light source, intensity modulated at one or more frequencies, will be referred to herein as the “Frequency Domain Photoacoustic” (FD-PA) technique. Although the FD-PA technique may include using frequency domain analysis, the technique may use time domain analysis, wavelet domain analysis, or any other suitable analysis, or any combination thereof. Accordingly, the term “frequency domain” as used in “FD-PA” refers to the frequency modulation of the photonic signal, and not to the type of analysis used to process the photoacoustic response.

Under some conditions, the acoustic pressure p(R,t) at detector position R at time t, may be shown illustratively by Eq. 5:

$$p ( R , t ) ∼ \frac{{p}_{0} ( {r}_{0} , ω )}{R} {e}^{- i ω ( t - τ )} \qquad ( 5 )$$

where r₀ is the position of the illuminated region of interest, ω is the angular frequency of the acoustic wave (caused by modulation of the photonic signal at frequency ω), R is the distance between the illuminated region of interest and the detector, and r is the travel time delay of the wave equal to R/cₐ, where cₐ is the speed of sound in the tissue. The FD-PA spectrum p₀(r₀,ω) of acoustic waves is shown illustratively by Eq. 6:

$${p}_{0} ( {r}_{0} , ω ) = \frac{{Γμ}_{a} φ ( {r}_{0} )}{2 ( {μ}_{a} {c}_{a} - i ω )} \qquad ( 6 )$$

where μₐcₐ represents a characteristic frequency (and corresponding time scale) of the tissue.

In some embodiments, a FD-PA system may temporally vary the characteristic modulation frequency of the CW light source, and accordingly the characteristic frequency of the associated acoustic response. For example, the FD-PA system may use linear frequency modulation (LFM), either increasing or decreasing with time, which is sometimes referred to as “chirp” signal modulation. Shown in Eq. 7 is an illustrative expression for a sinusoidal chirp signal r(t):

$$r ( t ) = sin ( t ( {ω}_{0} + \frac{b}{2} t ) ) \qquad ( 7 )$$

where ω₀ is a starting angular frequency, and b is the angular frequency scan rate. Any suitable range of frequencies (and corresponding angular frequencies) may be used for modulation such as, for example, 1-5 MHz, 200-800 kHz, or other suitable range, in accordance with the present disclosure. In some embodiments, signals having a characteristic frequency that changes as a nonlinear function of time may be used. Any suitable technique, or combination of techniques thereof, may be used to analyze a FD-PA signal. Two such exemplary techniques, a correlation technique and a heterodyne mixing technique, will be discussed below as illustrative examples.

In some embodiments, the correlation technique may be used to determine the travel time delay of the FD-PA signal. In some embodiments, a matched filtering technique may be used to process a PA signal. As shown in Eq. 8:

$${B}_{s} ( t - τ ) = \frac{1}{2 π } {\int }_{- ∞}^{∞} H ( ω ) S ( ω ) {e}^{i ω t} d ω \qquad ( 8 )$$

Fourier transforms (and inverse transforms) are used to calculate the filter output Bₛ(t−T), in which H(ω) is the filter frequency response, S(ω) is the Fourier transform of the PA signal s(t), and T is the phase difference between the filter and signal. In some circumstances, the filter output of expression of Eq. 8 may be equivalent to an autocorrelation function. Shown in Eq. 9:

$$S ( ω ) = \frac{1}{2 π } {\int }_{- ∞}^{∞} s ( t ) {e}^{- i ω t} d t \qquad ( 9 )$$

is an expression for computing the Fourier transform S(ω) of the PA signal s(t). Shown in Eq. 10:

H(ω)=S*(ω)e^{-iωτ} (10)

is an expression for computing the filter frequency response H(ω) based on the Fourier transform of the PA signal s(t). It can be observed that the filter frequency response of Eq. 10 requires the frequency character of the PA signal be known beforehand to determine the frequency response of the filter. In some embodiments, as shown by Eq. 11:

$$B ( t ) = {\int }_{- ∞}^{∞} r ( {t}^{′} ) s ( t + {t}^{′} ) d {t}^{′} \qquad ( 11 )$$

the known modulation signal r(t) may be used for generating a cross-correlation with the PA signal. The cross-correlation output B(t) of Eq. 11 is expected to exhibit a peak at a time t equal to the acoustic signal travel time τ. Assuming that the temperature response and resulting acoustic response follow the illumination modulation (e.g., are coherent), Eq. 11 may allow calculation of the time delay, depth information, or both.

In some embodiments, the heterodyne mixing technique may be used to determine the travel time delay of the FD-PA signal. The FD-PA signal, as described above, may have similar frequency character as the modulation signal (e.g., coherence), albeit shifted in time due to the travel time of the acoustic signal. For example, a chirp modulation signal, such as r(t) of Eq. 7, may be used to modulate a CW light source. Heterodyne mixing uses the trigonometric identity of the following Eq. 12:

$$sin ( A ) sin ( B ) = \frac{1}{2} [ cos ( A - B ) - cos ( A + B ) ] \qquad ( 12 )$$

which shows that two signals may be combined by multiplication to give periodic signals at two distinct frequencies (i.e., the sum and the difference of the original frequencies). If the result is passed through a low-pass filter to remove the higher frequency term (i.e., the sum), the resulting filtered, frequency shifted signal may be analyzed. For example, Eq. 13 shows a heterodyne signal L(t):

$$\begin{matrix}L ( t ) = 〈 r ( t ) s ( t ) 〉 \\ ≅ 〈 K r ( t ) r ( t - \frac{R}{{c}_{a}} ) 〉 \\ = \frac{1}{2} K cos ( \frac{R}{{c}_{a}} b t + θ )\end{matrix} \qquad ( 13 )$$

calculated by low-pass filtering (shown by angle brackets) the product of modulation signal r(t) and PA signal s(t). If the PA signal is assumed to be equivalent to the modulation signal, with a time lag R/cₐ due to travel time of the acoustic wave and amplitude scaling K, then a convenient approximation of Eq. 13 may be made, giving the rightmost term of Eq. 13. Analysis of the rightmost expression of Eq. 13 may provide depth information, travel time, or both. For example, a fast Fourier transform (FFT) may be performed on the heterodyne signal, and the frequency associated with the highest peak may be considered equivalent to time lag Rb/cₐ. Assuming that the frequency scan rate b and the speed of sound cₐ are known, the depth R may be estimated.

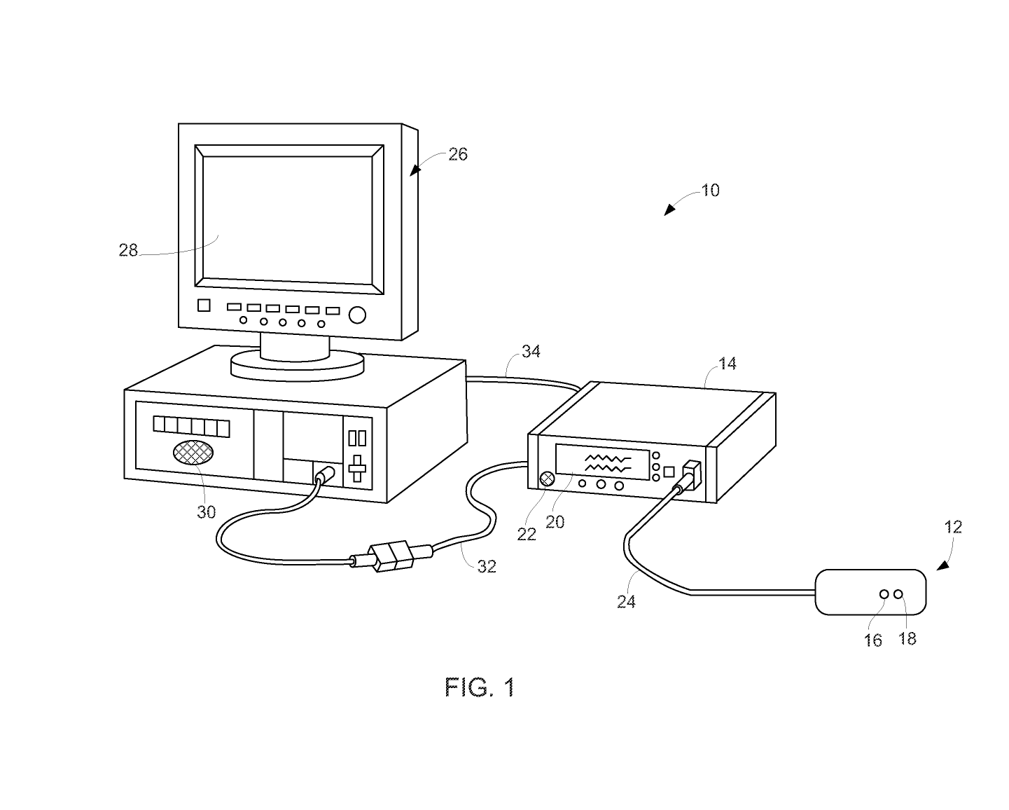

FIG. 1 is a perspective view of an embodiment of a physiological monitoring system 10. System 10 may include sensor unit 12 and monitor 14. In some embodiments, sensor unit 12 may be part of a photoacoustic monitor or imaging system. Sensor unit 12 may include a light source 16 for emitting light at one or more wavelengths into a subject's tissue. A detector 18 may also be provided in sensor unit 12 for detecting the acoustic (e.g., ultrasound) response that travels through the subject's tissue. Any suitable physical configuration of light source 16 and detector 18 may be used. In an embodiment, sensor unit 12 may include multiple light sources and/or acoustic detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body.

In some embodiments, system 10 may include two or more sensors forming a sensor array in lieu of either or both of the sensor units. In some embodiments, a sensor array may include multiple light sources, detectors, or both. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data relating to light emission and acoustic detection received from one or more sensor units such as sensor unit 12. For example, monitor 14 may be configured to determine pulse rate, blood pressure, blood oxygen saturation (e.g., arterial, venous, or both), hemoglobin concentration (e.g., oxygenated, deoxygenated, and/or total), any other suitable physiological parameters, or any combination thereof. In some embodiments, calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a subject's physiological parameters are not within a predefined normal range. In some embodiments, the system 10 includes a stand-alone monitor in communication with the monitor 14 via a cable or a wireless network link.

In some embodiments, sensor unit 12 may be communicatively coupled to monitor 14 via a cable 24. Cable 24 may include electronic conductors (e.g., wires for transmitting electronic signals from detector 18), optical fibers (e.g., multi-mode or single-mode fibers for transmitting emitted light from light source 16), any other suitable components, any suitable insulation or sheathing, or any combination thereof. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may be allow communication between monitor 14 and sensor unit 12.

In the illustrated embodiment, system 10 includes a multi-parameter physiological monitor 26. The monitor 26 may include a cathode ray tube display, a flat panel display (as shown) such as a liquid crystal display (LCD) or a plasma display, or may include any other type of monitor now known or later developed. Multi-parameter physiological monitor 26 may be configured to calculate physiological parameters and to provide a display 28 for information from monitor 14 and from other medical monitoring devices or systems (not shown). For example, multi-parameter physiological monitor 26 may be configured to display an estimate of a subject's blood oxygen saturation, hemoglobin concentration, pulse rate, and/or blood pressure generated by monitor 14. Multi-parameter physiological monitor 26 may include a speaker 30.

Monitor 14 may be communicatively coupled to multi-parameter physiological monitor 26 via a cable 32 or 34 that is coupled to a sensor input port or a digital communications port, respectively and/or may communicate wirelessly (not shown). In addition, monitor 14 and/or multi-parameter physiological monitor 26 may be coupled to a network to enable the sharing of information with servers or other workstations (not shown). Monitor 14 may be powered by a battery (not shown) or by a conventional power source such as a wall outlet.

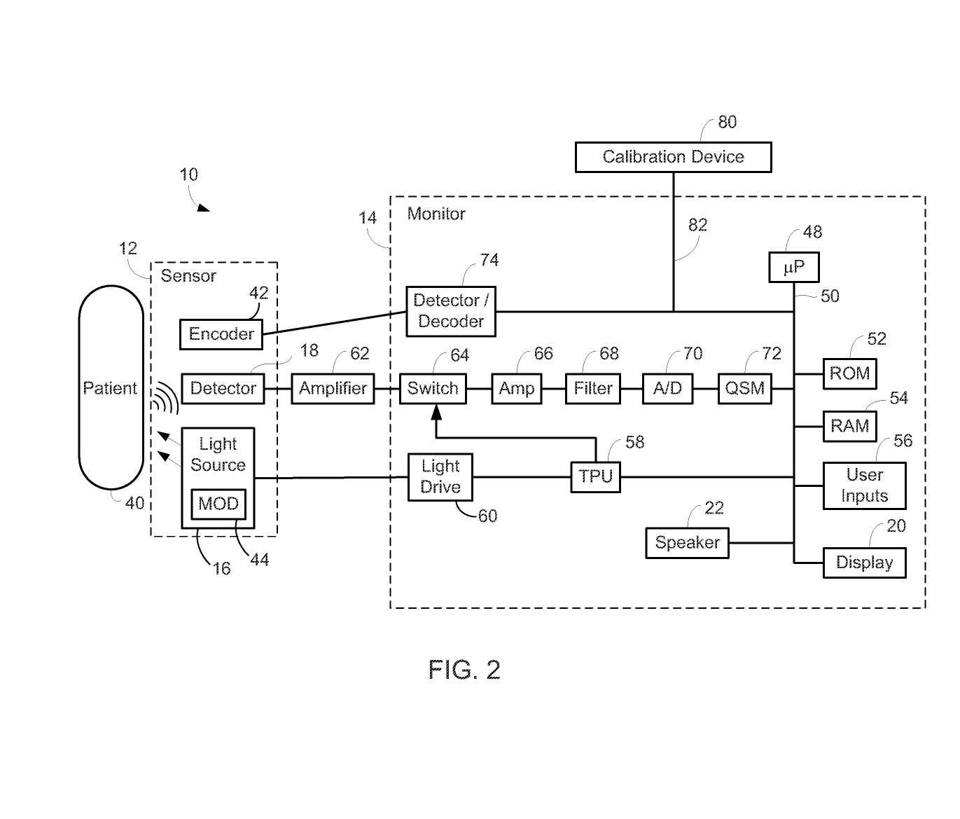

FIG. 2 is a block diagram of a physiological monitoring system, such as physiological monitoring system 10 of FIG. 1, which may be coupled to a subject 40 in accordance with an embodiment. Certain illustrative components of sensor unit 12 and monitor 14 are illustrated in FIG. 2.

Sensor unit 12 may include light source 16, detector 18, and encoder 42. In some embodiments, light source 16 may be configured to emit one or more wavelengths of light (e.g., visible, infrared) into a subject's tissue 40. Hence, light source 16 may provide Red light, IR light, any other suitable light, or any combination thereof, that may be used to calculate the subject's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to provide light of a single wavelength. For example, a first sensor may emit only a Red light while a second may emit only an IR light. In a further example, the wavelengths of light used may be selected based on the specific location of the sensor.

It will be understood that, as used herein, the term “light” may refer to energy produced by electromagnetic radiation sources. Light may be of any suitable wavelength and intensity, and modulations thereof, in any suitable shape and direction. Detector 18 may be chosen to be specifically sensitive to the acoustic response of the subject's tissue arising from use of light source 16. It will also be understood that, as used herein, the “acoustic response” shall refer to pressure and changes thereof caused by a thermal response (e.g., expansion and contraction) of tissue to light absorption by the tissue or constituent thereof.

In some embodiments, detector 18 may be configured to detect the acoustic response of tissue to the photonic excitation caused by the light source. In some embodiments, detector 18 may be a piezoelectric transducer which may detect force and pressure and output an electrical signal via the piezoelectric effect. In some embodiments, detector 18 may be a Faby-Perot interferometer, or etalon. For example, a thin film (e.g., composed of a polymer) may be irradiated with reference light, which may be internally reflected by the film. Pressure fluctuations may modulate the film thickness, thus causing changes in the reference light reflection which may be measured and correlated with the acoustic pressure. In some embodiments, detector 18 may be configured or otherwise tuned to detect acoustic response in a particular frequency range. Detector 18 may convert the acoustic pressure signal into an electrical signal (e.g., using a piezoelectric material, photodetector of a Faby-Perot interferometer, or other suitable device). After converting the received acoustic pressure signal to an electrical signal, detector 18 may send the signal to monitor 14, where physiological parameters may be calculated based on the photoacoustic activity within the subject's tissue 40.

In some embodiments, encoder 42 may contain information about sensor unit 12, such as what type of sensor it is (e.g., where the sensor is intended to be placed on a subject), the wavelength(s) of light emitted by light source 16, the intensity of light emitted by light source 16 (e.g., output wattage or Joules), the mode of light source 16 (e.g., pulsed versus CW), any other suitable information, or any combination thereof. This information may be used by monitor 14 to select appropriate algorithms, lookup tables and/or calibration coefficients stored in monitor 14 for calculating the subject's physiological parameters.

Encoder 42 may contain information specific to subject 40, such as, for example, the subject's age, weight, and diagnosis. This information about a subject's characteristics may allow monitor 14 to determine, for example, subject-specific threshold ranges in which the subject's physiological parameter measurements should fall and to enable or disable additional physiological parameter algorithms. Encoder 42 may, for instance, be a coded resistor that stores values corresponding to the type of sensor unit 12 or the type of each sensor in the sensor array, the wavelengths of light emitted by light source 16 on each sensor of the sensor array, and/or the subject's characteristics. In some embodiments, encoder 42 may include a memory on which one or more of the following information may be stored for communication to monitor 14: the type of the sensor unit 12; the wavelengths of light emitted by light source 16; the particular acoustic range that each sensor in the sensor array is monitoring; a signal threshold for each sensor in the sensor array; any other suitable information; or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control the activation of light source 16. For example, TPU 58 may control pulse timing (e.g., pulse duration and inter-pulse interval) for TO-PA monitoring system. TPU 58 may also control the gating-in of signals from detector 18 through amplifier 62 and switching circuit 64. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as processing equipment.

In the embodiment shown, light source 16 may include modulator 44, in order to, for example, perform FD-PA analysis. Modulator 44 may be configured to provide intensity modulation, spatial modulation, any other suitable optical signal modulations, or any combination thereof. For example, light source 16 may be a CW light source, and modulator 44 may provide intensity modulation of the CW light source such as using a linear sweep modulation. In some embodiments, modulator 44 may be included in light drive 60, or other suitable components of physiological monitoring system 10, or any combination thereof.

In some embodiments, microprocessor 48 may determine the subject's physiological parameters, such as SpO₂, SvO₂, oxy-hemoglobin concentration, deoxy-hemoglobin concentration, total hemoglobin concentration (tHb), and/or pulse rate, using various algorithms and/or lookup tables based on the value of the received signals and/or data corresponding to the acoustic response received by detector 18. Signals corresponding to information about subject 40, and particularly about the acoustic signals emanating from a subject's tissue over time, may be transmitted from encoder 42 to decoder 74. These signals may include, for example, encoded information relating to subject characteristics. Decoder 74 may translate these signals to enable the microprocessor to determine the thresholds based at least in part on algorithms or lookup tables stored in ROM 52. In some embodiments, user inputs 56 may be used enter information, select one or more options, provide a response, input settings, any other suitable inputting function, or any combination thereof. User inputs 56 may be used to enter information about the subject, such as age, weight, height, diagnosis, medications, treatments, and so forth. In some embodiments, display 20 may exhibit a list of values, which may generally apply to the subject, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

The acoustic signal attenuated by the tissue of subject 40 can be degraded by noise, among other sources. Movement of the subject may also introduce noise and affect the signal. For example, the contact between the detector and the skin, or the light source and the skin, can be temporarily disrupted when movement causes either to move away from the skin. Another source of noise is electromagnetic coupling from other electronic instruments.

Noise (e.g., from subject movement) can degrade a sensor signal relied upon by a care provider, without the care provider's awareness. This is especially true if the monitoring of the subject is remote, the motion is too small to be observed, or the care provider is watching the instrument or other parts of the subject, and not the sensor site. Processing sensor signals may involve operations that reduce the amount of noise present in the signals, control the amount of noise present in the signal, or otherwise identify noise components in order to prevent them from affecting measurements of physiological parameters derived from the sensor signals.

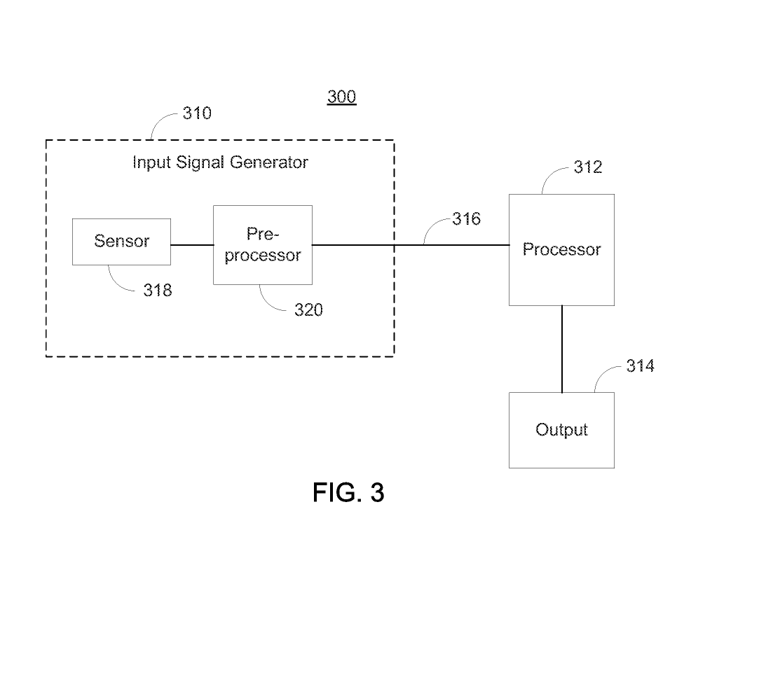

FIG. 3 is an illustrative signal processing system 300 in accordance with an embodiment that may implement the signal processing techniques described herein. In some embodiments, signal processing system 300 may be included in a physiological monitoring system (e.g., physiological monitoring system 10 of FIGS. 1-2). In the illustrated embodiment, input signal generator 310 generates an input signal 316. As illustrated, input signal generator 310 may include pre-processor 320 coupled to sensor 318, which may provide input signal 316. In some embodiments, pre-processor 320 may be a photoacoustic module and input signal 316 may be a photoacoustic signal. In an embodiment, pre-processor 320 may be any suitable signal processing device and input signal 316 may include one or more photoacoustic signals and one or more other physiological signals, such as a photoplethysmograph signal. It will be understood that input signal generator 310 may include any suitable signal source, signal generating data, signal generating equipment, or any combination thereof to produce signal 316. Signal 316 may be a single signal, or may be multiple signals transmitted over a single pathway or multiple pathways.

Pre-processor 320 may apply one or more signal processing operations to the signal generated by sensor 318. For example, pre-processor 320 may apply a pre-determined set of processing operations to the signal provided by sensor 318 to produce input signal 316 that can be appropriately interpreted by processor 312, such as performing A/D conversion. In some embodiments, A/D conversion may be performed by processor 312. Pre-processor 320 may also perform any of the following operations on the signal provided by sensor 318: reshaping the signal for transmission, multiplexing the signal, modulating the signal onto carrier signals, compressing the signal, encoding the signal, and filtering the signal.

In some embodiments, signal 316 may be coupled to processor 312. Processor 312 may be any suitable software, firmware, hardware, or combination thereof for processing signal 316. For example, processor 312 may include one or more hardware processors (e.g., integrated circuits), one or more software modules, and computer-readable media such as memory, firmware, or any combination thereof. Processor 312 may, for example, be a computer or may be one or more chips (i.e., integrated circuits). Processor 312 may, for example, include an assembly of analog electronic components. Processor 312 may calculate physiological information. For example, processor 312 may perform time domain calculations, spectral domain calculations, time-spectral transformations (e.g., fast Fourier transforms, inverse fast Fourier transforms), any other suitable calculations, or any combination thereof. Processor 312 may perform any suitable signal processing of signal 316 to filter signal 316, such as any suitable band-pass filtering, adaptive filtering, closed-loop filtering, any other suitable filtering, and/or any combination thereof. Processor 312 may also receive input signals from additional sources (not shown). For example, processor 312 may receive an input signal containing information about treatments provided to the subject. Additional input signals may be used by processor 312 in any of the calculations or operations it performs in accordance with processing system 300.

In some embodiments, all or some of pre-processor 320, processor 312, or both, may be referred to collectively as processing equipment. For example, processing equipment may be configured to amplify, filter, sample and digitize signal 316 (e.g., using an analog to digital converter), and calculate physiological information from the digitized signal.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. In some embodiments, processor 312 may store physiological measurements or previously received data from signal 316 in a memory device for later retrieval. In some embodiments, processor 312 may store calculated values, such as pulse rate, blood pressure, blood oxygen saturation (e.g., arterial, venous, or both), hemoglobin concentration (e.g., oxygenated, deoxygenated, and/or total), or any other suitable calculated values, in a memory device for later retrieval.

Processor 312 may be coupled to output 314. Output 314 may be any suitable output device such as one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIGS. 1 and 2) in which, for example, input signal generator 310 may be implemented as part of sensor unit 12 (FIGS. 1 and 2) and monitor 14 (FIGS. 1 and 2) and processor 312 may be implemented as part of monitor 14 (FIGS. 1 and 2). In some embodiments, portions of system 300 may be configured to be portable. For example, all or part of system 300 may be embedded in a small, compact object carried with or attached to the subject (e.g., a watch, other piece of jewelry, or a smart phone). In some embodiments, a wireless transceiver (not shown) may also be included in system 300 to enable wireless communication with other components of system 10 (FIGS. 1 and 2). As such, system 10 (FIGS. 1 and 2) may be part of a fully portable and continuous physiological monitoring solution. In some embodiments, a wireless transceiver (not shown) may also be included in system 300 to enable wireless communication with other components of system 10. For example, pre-processor 320 may output signal 316 (e.g., which may be a pre-processed photoacoustic signal) over BLUETOOTH, IEEE 802.11, WiFi, WiMax, cable, satellite, Infrared, any other suitable transmission scheme, or any combination thereof. In some embodiments, a wireless transmission scheme may be used between any communicating components of system 300.

It will also be understood that while some of the equations referenced herein are continuous functions, the processing equipment may be configured to use digital or discrete forms of the equation in processing the acquired PA signal.

The PA signal obtained by system 10 or 300 is dependent on the optical fluence at the illuminated region of interest, as shown in Eq. 1, for example. While, the output of the light source may be modulated, measured, regulated, or otherwise controlled, the resulting light output may be attenuated along its pathlength prior to illumination of the region of interest. Accurately estimating the optical fluence at the region of interest may improve the accuracy of the resulting PA calculations.

In some embodiments, the illuminated region of interest may include a blood vessel such as an artery, vein, or capillary, for example. The blood within the vessel may absorb a portion of the incident optical fluence at the vessel. The resulting acoustic pressure signal may exhibit two sequential peaks (in the time domain) generated primarily from the boundary between the blood and the adjacent tissue (e.g., a blood vessel). The acoustic pressure signal, as detected at a suitable detector, may be greater when that boundary surface faces the detector. The first peak may be indicative of the front boundary between the blood and the vessel (relatively closer to the light source), and the second peak may be indicative of the back boundary between the blood and the vessel (relatively further from the light source).

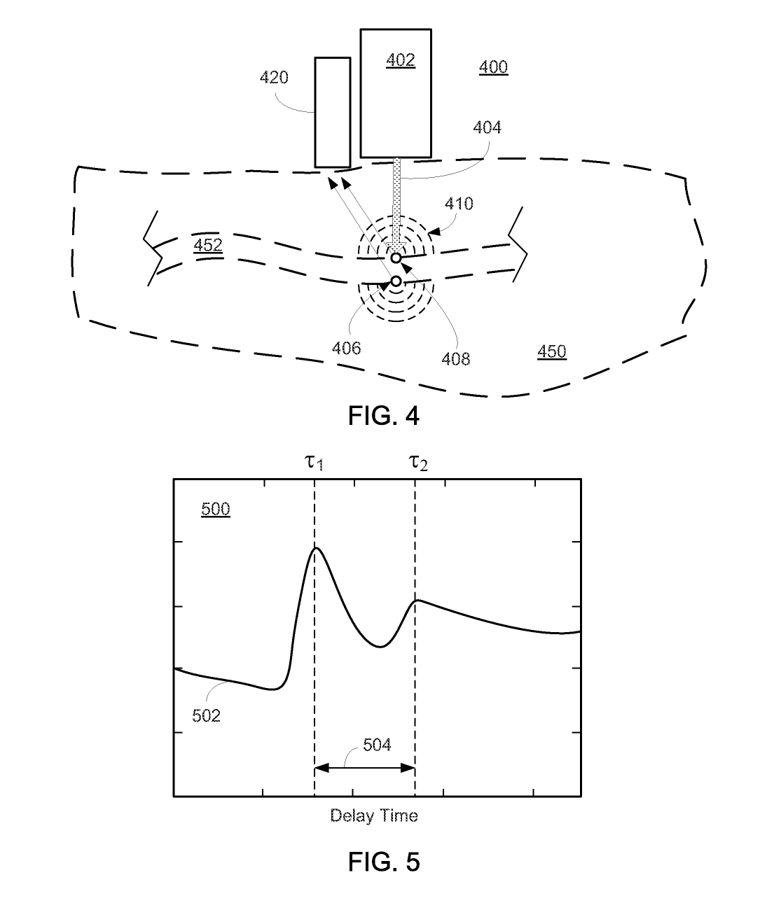

FIG. 4 is an illustrative photoacoustic arrangement 400, in accordance with some embodiments of the present disclosure. Light source 402, controlled by a suitable light drive (e.g., a light drive of system 300 or system 10, although not shown in FIG. 4), may provide photonic signal 404 to subject 450. Photonic signal 404 may be attenuated along it's pathlength in subject 450 prior to reaching blood vessel 452. A constituent of the blood in blood vessel 452 such as, for example, hemoglobin, may absorb at least some of photonic signal 404. Accordingly, the blood may exhibit an acoustic pressure response via the photoacoustic effect, which may act on the boundary of blood vessel 452. Acoustic pressure signals 410 may travel through subject 450, originating substantially from the front boundary 408 and back boundary 406 of blood vessel 452. Acoustic detector 420 may detect acoustic pressure signals 410 traveling through tissue of subject 450, and output (not shown) a photoacoustic signal that may be processed. Because the path length between point 408 and acoustic detector 420 is shorter than the pathlength between point 406 and acoustic detector 420, it may be expected that acoustic pressure signals from point 408 may reach acoustic detector 420 before acoustic pressure signals from point 406. Additionally, in some arrangements, because the path length between point 408 and acoustic detector 420 is shorter than the path length between point 406 and acoustic detector 420, it may be expected that an acoustic pressure signal from point 408 may exhibit a relatively larger peak than an acoustic pressure signal from point 406. Accordingly, acoustic detector 420 may detect two sequential peaks in acoustic pressure signal 410 generated by photonic signal 404 directed at blood vessel 452.

FIG. 5 is a plot 500 of an illustrative photoacoustic signal 502, including two peaks, in accordance with some embodiments of the present disclosure. The abscissa of plot 500 is presented in units proportional to time (e.g., delay time relative to a light pulse), while the ordinate of plot 500 is presented in arbitrary units of signal intensity. At least a portion of photoacoustic signal 502 corresponds to the acoustic pressure response of blood within a blood vessel. Photoacoustic signal 502 exhibits a first peak and a second peak, located at respective times τ₁ and τ₂. The first peak corresponds to the front boundary of the blood vessel, relatively nearer to the acoustic detector. The second peak corresponds to the back boundary of the blood vessel, relatively further from the acoustic detector. Time difference 504 between τ₁ and τ₂ indicates the relative difference in delay time between acoustic pressure signals from the front and back boundaries. In some embodiments, comparison of the first and second peaks may allow determination of one or more physiological parameters.

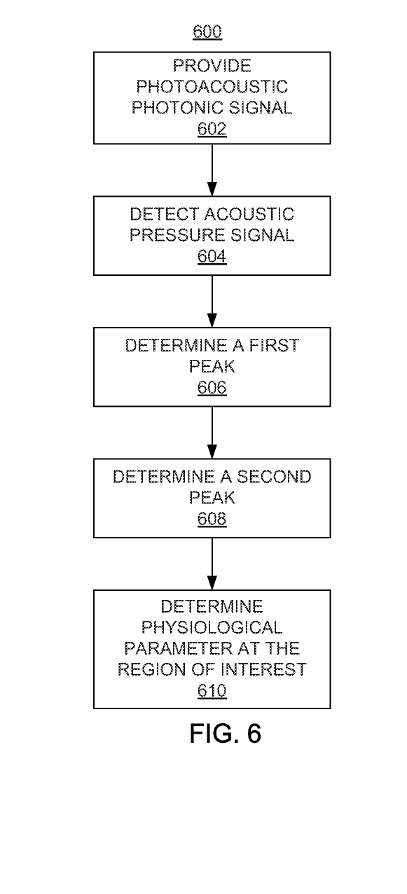

FIG. 6 is a flow diagram 600 of illustrative steps for determining a physiological parameter based at least in part on two peaks of an acoustic pressure signal, in accordance with some embodiments of the present disclosure.

Step 602 may include a suitable light source (e.g., light source 16 of system 10) of system 300 providing a photonic signal to a subject. The light source may be a pulsed light source, continuous wave light source, any other suitable light source, or any combination thereof. In some embodiments, modulator 44 may be used to modulate the photonic signal of the light source. In some embodiments, the photonic signal may be focused or otherwise spatially modulated. For example, the photonic signal may be focused on or near a blood vessel, which may contain blood that absorbs at least some of the photonic signal, causing a relatively stronger photoacoustic response and accordingly a stronger photoacoustic signal.

Step 604 may include system 300 detecting an acoustic pressure signal. In some embodiments, an acoustic detector such as, for example, an ultrasound detector of system 300 may detect the acoustic pressure signal. The acoustic detector may output an electrical signal to suitable processing equipment of system 300. The acoustic pressure signal may be detected as a time series (e.g., in the time domain or sample number domain), and processed as a time series, as a spectral series (e.g., in the frequency domain), any other suitable series, or any combination thereof. In some embodiments, pre-processor 320 may pre-process the detected acoustic pressure signal. For example, pre-processor 320 may perform filtering, amplifying, de-multiplexing, de-modulating, sampling, smoothing, any other suitable pre-processing, or any combination thereof.

Step 606 may include system 300 determining a first peak of the detected acoustic pressure signal of step 604. In some embodiments, processor 312 may use a peak finding technique to determine the first peak. For example, processor 312 may locate a maximum in the photoacoustic signal, locate a zero in the first derivative of the photoacoustic signal, perform any other suitable peak finding technique, or any combination thereof. The peak finding technique may operate on only a subset of the photoacoustic signal. For example, the peak finding algorithm may only start looking for a peak after a predetermined time or sample number. The starting location may be determined based on the expected depth location of the blood vessel of interest. Determining a first peak may include determining a peak value, determining a peak location (e.g., in time or sample number), determining a peak property (e.g., full-width at half maximum, height to width ratio), comparing a property of a peak to a predetermined threshold (e.g., to qualify the peak), performing any other suitable determination, or any combination thereof.

Step 608 may include system 300 determining a second peak of the detected acoustic pressure signal of step 604. In some embodiments, processor 312 may use a peak finding technique to determine the second peak. For example, processor 312 may locate a maximum in the photoacoustic signal, locate a zero in the first derivative of the photoacoustic signal, use the determined first peak to aid in locating the second peak (e.g., use a relative time value), perform any other suitable peak finding technique, or any combination thereof. Determining a second peak may include determining a peak value, determining a peak location (e.g., in time or sample number), determining a peak property (e.g., full-width at half maximum, height to width ratio), comparing a property of a peak to a predetermined threshold (e.g., to qualify the peak), performing any other suitable determination, or any combination thereof.

Step 610 may include system 300 determining one or more physiological parameters of the subject based at least in part on the first and second peaks. Physiological parameters may include pulse rate, hemoglobin concentration (e.g., deoxy-hemoglobin, oxyhemoglobin, or total), blood oxygen saturation (e.g., arterial, venous), any other suitable physiological parameters, any physiological modulations thereof, or any combination thereof. For example, in some circumstances, a subject's pulse rate may modulate the time interval between peaks (e.g., by modulating blood vessel properties), which may be detectable by system 300. In a further example, one or more optical properties of a subject may be determined based on the first and second peaks of the photoacoustic signal. The one or more optical properties may be used to determine one or more physiological properties based on mathematical correlations, models, or both. For example, an effective attenuation coefficient may be correlated to oxy-hemoglobin concentration, deoxy-hemoglobin concentration, total hemoglobin concentration, or blood oxygen saturation (e.g., arterial or venous depending on a blood vessel type).

The following discussion provides an illustrative example of the steps of flow diagram 600, in accordance with some embodiments of the present disclosure. In this illustrative example, the photonic signal is a pulsed signal, generated by a suitable pulsed light source of system 300. The photonic signal may include any wavelength suitable for the interrogated feature (e.g., a blood vessel or other feature), at any suitable spatial resolution. For example, regarding a blood vessel with a nominal diameter of two millimeters, a spatial resolution of 0.2 millimeters may be used in accordance with the disclosed techniques. The photonic signal may be absorbed in part by a constituent of a subject's blood within a blood vessel. The optical fluence may vary across the blood vessel. Absorption of the portion of the photonic signal may cause a photoacoustic response, and corresponding peaks in acoustic pressure from the front and back boundaries of the blood vessel at respective first and second times. The photoacoustic signal, at the first and second times, is given by the following respective equations:

P(τ₁)=Γμₐφ₀e^{-μ}^{_{eff}}^{Cτ}^{₁} (14)

P(τ₂)=Γμₐφ₀(e^{-μ}^{_{eff}}^{Cτ}^{₁})(e^{-μ}^{ₑ}^{C(τ}^{₂}^{-τ}^{₁}⁾), (15)

in which P(τ) is the PA signal at time τ, Γ is the dimensionless Grüneisen parameter of the tissue, μₐ is the effective absorption coefficient of the tissue (or constituent thereof such as hemoglobin in this example) to the incident light, φ₀ is the optical fluence at the tissue surface, c is the speed of sound in the tissue, μ_{eff} is the effective attenuation coefficient of the tissue along the optical path length in the tissue in this example, and μₑ is the effective attenuation coefficient of the blood within the blood vessel. It will be understood that the functional form of the optical fluence attenuation as a decaying exponential function is an illustrative model, and any suitable dependency may be used in accordance with the present disclosure. The effective attenuation coefficient may be formulated as follows:

μ^{e}=\sqrt{3μₐ(μₐ+μ′ₛ)} (16)

in which, μ′ₛ is the reduced scattering coefficient of the tissue (or constituent thereof such as hemoglobin in this example).

The expression of Eq. 14 may be divided by the expression of Eq. 15, creating the following equation:

$$\begin{matrix}\frac{P ( {τ}_{1} )}{P ( {τ}_{2} )} = \frac{{Γμ}_{a} {φ}_{0} {e}^{- {μ}_{eff} c {τ}_{1}}}{{Γμ}_{a} {φ}_{0} ( {e}^{- {μ}_{eff} {cτ}_{1}} ) ( {e}^{- {μ}_{e} c ( {τ}_{2} - {τ}_{1} )} )} \\ = {e}^{{μ}_{e} ( c ( {τ}_{2} - {τ}_{1} ) )} ,\end{matrix} \qquad ( 17 )$$

in which the Grüneisen parameter, the effective absorption coefficient of the tissue, and the optical fluence at the tissue surface are mathematically cancelled. The resulting Eq. 17 may be applied to an acoustic pressure response signal, in which τ₁ is a time corresponding to a first peak, and τ₂ is a time corresponding to a second peak. Assuming the speed of sound is known, Eq. 17 may be solved for the effective absorption coefficient. The effective attenuation coefficient, and more particularly the absorption coefficient and reduced scattering coefficient, may be dependent on hemoglobin concentration, and may accordingly be correlated with a physiological parameter. In some embodiments, the wavelength of the light source may be selected to aid in determining one or more physiological parameters. For example, at a first wavelength λ₁ where oxy-hemoglobin and deoxy-hemoglobin have approximately the same absorptivity (e.g., around 808 nm), the absorption coefficient μ_{a,λ}_{₁} and reduced scattering coefficient μ′_{s,λ}_{₁} may be given the following:

μ_{a,λ}_{₁}=tHb·ε_{λ}_{₁}, (18)

μ′_{s,λ}_{₁}=f(tHb,λ₁), (19)

where ε_{λ}_{₁} (presumed known) is the absorptivity of the oxy-hemoglobin and deoxy-hemoglobin at first wavelength λ₁. The reduced scattering coefficient may, in some embodiments, be a polynomial function of tHb such as, for example, a second or third order polynomial for which the coefficients may depend on λ₁. Using Eqs. 18-19 in Eq. 16, the known effective attenuation coefficient can be cast in terms of tHb as shown by:

μₑ=f(tHb), (20)

which may be inverted to determine tHb from the known μ_{e,λ}_{₁}. In some embodiments, a second light source of a second wavelength λ₂, different from the first, may be used to determine blood oxygen saturation. For example, with tHb known, a second effective attenuation coefficient may be determined at the second wavelength. The absorption coefficient μₐ and reduced scattering coefficient μ′_{s,λ}_{₂} at λ₂ may be given by the following:

μ_{a,λ}_{₂}=ε_{ox,λ}_{₂}cₒₓ+ε_{deox,λ}_{₂}c_{deox}, (21)

μ′_{s,λ}_{₂}=f(cₒₓ,c_{deox},λ₂), (22)

where ε_{ox,λ}_{₂} is the absorptivity of oxy-hemoglobin, ε_{deox,λ}_{₂} is the absorptivity of deoxy-hemoglobin, cₒₓ is the concentration of oxy-hemoglobin, and c_{deox} is the concentration of deoxy-hemoglobin. The concentration can be related by:

tHb=cₒₓ+c_{deox}, (23)

which may be combined with Eq.16 and Eqs. 21-22 to give:

μ_{e,λ}_{₂}=f(tHb,cₒₓ), or (24)

μ_{e,λ}_{₂}=f(tHb,c_{deox}). (25)

Because tHb is known, any of Eqs. 24 and 25 may be inverted to determine the respective hemoglobin concentration from the known tHb and μ_{e,λ}_{₂}. Additionally, blood oxygen saturation S_{O2} may be determined by the following:

$${S}_{O 2} = \frac{{c}_{ox}}{{c}_{ox} + {c}_{deox}} , \qquad ( 26 )$$

which may be an arterial blood oxygen saturation or venous oxygen saturation depending upon the type of blood vessel. It will be understood that Eqs. 16-26 provide illustrative examples of formulas used to determine physiological parameters from photoacoustic measurements. Any suitable equations, models, other suitable mathematical construct, look-up table, database, or other reference may be used to determine one or more physiological parameters based on two photoacoustic peaks. For example, in some embodiments, physiological parameters may be tabulated (e.g., in a look-up table stored in encoder 42 of system 10) for discrete values of effective attenuation coefficient at one or more wavelengths.

In a further illustrative example of the steps of flow diagram 600, a FD-PA analysis may be performed on the photoacoustic signal. The correlation technique, as shown by Eq. 11, includes performing a cross-correlation of a photoacoustic signal s(t) with a known modulation signal r(t). In this illustrative example, the modulation signal is a chirp signal, which modulates a CW photonic signal. The photonic signal, provided by a light source of system 300, may be absorbed in part by a constituent of a subject's blood within a blood vessel. Absorption of the portion of the photonic signal may cause a photoacoustic response, and corresponding peaks in acoustic pressure from the front and back boundaries of the blood vessel. The detected photoacoustic signal may exhibit a superposition of the acoustic pressures generated at the front and back boundaries, offset in time due to the travel delay. Accordingly, the cross-correlation of the photoacoustic signal and the modulation signal may exhibit two peaks; the first peak corresponding to the front boundary, and the second peak corresponding to the back boundary. The time difference between the first and second peaks may be inputted into a suitable expression such as, for example, Eq. 17, and one or more optical properties of the subject may be solved for. The one or more optical properties may accordingly be correlated with a physiological parameter.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A method for monitoring the photoacoustic response of a subject to determine a physiological parameter, the method comprising:
providing a photonic signal to the subject;
detecting an acoustic pressure signal at one or more locations of the subject using one or more acoustic detectors, wherein the acoustic pressure signal is caused by the absorption of at least some of the photonic signal by the subject;
determining a first peak in the acoustic pressure signal indicative of a first boundary of a feature within the subject;
determining a second peak in the acoustic pressure signal indicative of a second boundary of the feature; and
determining a physiological parameter of the subject based at least in part on the first and second peaks.

2. The method of claim 1, wherein the feature is a blood vessel of the subject.

3. The method of claim 2, wherein the determining the physiological parameter of the subject further comprises determining one or more of arterial blood oxygen saturation, venous blood oxygen saturation, and total hemoglobin concentration.

4. The method of claim 1, wherein the determining the first peak comprises determining a first time associated with the first peak, and wherein the determining the second peak comprises determining a second time associated with the second peak, and wherein the determining the physiological parameter comprises determining a difference between the second time and the first time.

5. The method of claim 4, wherein the first time corresponds to a front boundary of the feature, and the second time corresponds to a back boundary of the feature.

6. The method of claim 4, wherein the first peak and the second peak occur sequentially, and wherein the first time is before the second time.

7. The method of claim 1, wherein the determining the physiological parameter of the subject comprises dividing the acoustic pressure signal at the first time by the acoustic pressure signal at the second time to generate a ratio.

8. The method of claim 7, further comprising determining an effective attenuation coefficient of the subject based at least in part on the generated ratio.

9. The method of claim 8, wherein the determining the physiological parameter of the subject further comprises determining the physiological parameter of the subject based at least in part on the effective attenuation coefficient.

10. The method of claim 1, wherein the providing the photonic signal to the subject comprises providing the photonic signal at a wavelength allowing the photonic signal to substantially penetrate the subject to reach the feature.

11. A physiological monitoring system for monitoring a subject, the system comprising:
a light source configured to provide a photonic signal to a feature of the subject;
an acoustic detector configured to detect the acoustic pressure signal at one or more locations of the subject, wherein the acoustic pressure signal is caused by the absorption of at least some of the photonic signal by the subject; and
a processor communicatively coupled to the acoustic detector, the processor configured to:
determine a first peak in the acoustic pressure signal indicative of a first surface of the feature,
determine a second peak in the acoustic pressure signal indicative of a second surface of the feature, and
determine a physiological parameter of the subject based at least in part on the first and second peaks.

12. The system of claim 11, wherein the processor is further configured to determine at least one parameter selected from the consisting of an arterial blood oxygen saturation value, a venous blood oxygen saturation value, a total hemoglobin concentration, and any combination thereof.

13. The system of claim 11, wherein the processor is further configured to:
determine a first time associated with the first peak,
determine a second time associated with the second peak, and
determine a difference between the second time and the first time.

14. The system of claim 11, wherein the processor is further configured to divide the acoustic pressure signal at the first time by the acoustic pressure signal at the second time to generate a ratio.

15. The system of claim 14, wherein the processor is further configured to determine an effective attenuation coefficient of the subject based at least in part on the generated ratio.

16. The system of claim 15, wherein the processor is further configured to determine the physiological parameter of the subject based at least in part on the effective attenuation coefficient.

17. The system of claim 11, wherein the light source is further configured to provide the photonic signal at a wavelength that substantially penetrates the subject to reach the feature.

18. The system of claim 11, wherein the light source is further configured to provide a pulsed photonic signal.

19. The system of claim 11, wherein the light source is further configured to provide a continuous wave photonic signal.

20. The system of claim 19, further comprising a modulator, wherein the modulator is further configured to modulate the intensity of the continuous wave photonic signal.
